(19) United States
(12) Patent Application Publication
(10) Pub. No.: US 2012/0123187 A1
(43) Pub. Date: May 17, 2012

(54) System and Method for Generating a Radiation Treatment Plan for Use in Effecting Radiation Therapy in a Human or Animal Body

(76) Inventors: Rob van der Laarse, Zeist (NL); Mehmet Uzumcu, Amersfoort (NL)

(21) Appl. No.: 13/377,491

(22) PCT Filed: Jun. 11, 2010
(86) PCT No.: PCT/NL2010/050358

§ 371 Date: Jan. 31, 2012

(30) Foreign Application Priority Data
Jun. 11, 2009 (NL) 1037032

(51) Int. Cl. A61M 36/04

(52) U.S. Cl. 600/3

(57) ABSTRACT

The invention relates to a method for generating a radiation treatment plan and a radiation therapy treatment planning system for use in effecting radiation therapy of an anatomical portion of an animal body. The invention also relates to a radiation therapy delivery system.

The present invention aims to provide a new method for generating a radiation treatment plan for use in effecting radiation therapy of an anatomical portion of a human and animal body, whereby a more efficient use can be made of the virtual working space of the treatment planning system and hence provide a more accurate match between the planned radiation dose distribution and the ideal radiation dose distribution profile position. This is achieved by defining a starting position and a finishing position along said trajectory for each of said catheters and by defining a dwell step corresponding to an equal number of dwell positions fitting the trajectory between said starting position and said finishing position and then determining the radiation dose distribution for each of said catheters based on said equal number of dwell positions.

The invention relates to a method for generating a radiation treatment plan for use in effecting radiation therapy of an anatomical portion of a human or animal body.

The invention also relates to a radiation therapy treatment planning system for use in effecting radiation therapy of a pre-selected anatomical portion of a human or animal body.

The invention also relates to a radiation therapy delivery system.

A system as mentioned in the preamble above is for example disclosed in the European patent application no. 1374949 in the name of the applicant of this patent application. In EP 1374949 a treatment plan is generated wherein energy emitting sources are displaced through several catheters already implanted into the patient body. Usually, for this purpose a source having length of about 3.7 mm is displaced in a step-wise fashion along a catheter with steps of a bout 5 mm for effectuating a pre-defined dose distribution around the catheter. It is a common practice to calculate a relative dose distribution about the catheter, which is normalized at a distance of 1 cm from a centre of the catheter.

In brachy therapy treatment applications, cancerous tissue, for example the male prostate gland, the female breast or nearly anywhere else in an animal or human body where tumours are accessible, is canalized with one or more hollow treatment catheters. The treatment catheters are connected outside the patient's body with a so-called after-loading apparatus having radiation delivery means for advancing one or more energy emitting sources through said catheters. In known treatment planning solutions generated prior to the treatment, the energy emitting source (or sources) are stopped at pre-defined positions within the catheter (and hence inside the treatment site) for pre-defined times.

In general the pre-defined positions are known as dwell positions, and the pre-defined times at which the energy emitting source are halted in a specific dwell position are known as dwell times. Dwell positions and dwell times are calculated in a treatment planning unit by discrete optimization algorithms. It is appreciated that a dwell step is selected based on a compromise between the allowable calculation time for the dose profile in 3D and a desired calculation grid. It is found that a dwell step of about 5 mm meets both accuracy criteria and the calculation speed. It is further found that the dwell step is relatively independent of the source length and the anisotropy of the source. Usually, a source having 3.7 mm length is used.

However treatment planning solutions containing amongst others a set of dwell positions and dwell times in the inserted catheters provide a discrete treatment solution. As an energy emitting source is stopped for a certain dwell time at each dwell position the radiation dose delivered in that position exhibits a point source like distribution of which the peak (or height) is determined by the length of the dwell time interval at said dwell position as well as other factors, such as the level of activity of the source.

Such a discrete treatment planning solution does not provide the ideal treatment planning solution, where it is intended that the target location receives a homogeneous dose coverage and where healthy tissue surrounding the target location is prevented from receiving radiation.

The discrete optimization techniques which are used to determine the radiation dose distributions for each catheter were first developed in the 1970s. These techniques when used to treat breast cancer and prostate cancer by means of implants made use of locally defined template grids to determine what the optimal solution was for the intended treatment. These optimization techniques matched well with the earliest brachytherapy treatment systems.

The use of a single step size for all catheters restricts the planning software in that the positioning of virtual catheters and energy emitting sources within the virtual anatomical portion to be treated is bound to and limited to these fixed dwell positions. This restricts the placement of the outer dwell position in each catheter in relation to the boundary of the target.

The present invention aims to provide a new method for generating a radiation treatment plan for use in effecting radiation therapy of an anatomical portion of an animal body, whereby a more efficient use can be made of the virtual working space of the treatment planning system and hence provides a more accurate match between the planned radiation dose distribution and the ideal radiation dose distribution profile position. It is a further object of the invention to provide a radiation planning system wherein homogeneity of the calculated dose distribution is substantially independent of a length of a trajectory of the source inside a catheter.

According to the invention there is provided a radiation therapy treatment planning system for use in effecting radiation therapy of a pre-selected anatomical portion of a human or animal body, whereby one or more catheters are inserted in a certain orientation into said anatomical portion, each catheter defining a trajectory for at least one energy emitting source to be displaced along said trajectory through said catheter said radiation treatment planning system comprising:

treatment planning means for generating a radiation treatment plan for effecting said radiation therapy, said treatment plan at least comprising information concerning:

the number, position and direction of said one or more of said catheters within said anatomical portion to be treated,

default dwell positions and dwell times for said at least one energy emitting source along respective trajectories defined for said catheters, each trajectory having a starting position and a finishing position for each of said one or more catheters, and

a radiation dose distribution for each of said one or more catheters, said treatment planning system including means to generate a treatment plan, arranged to determine a revised dwell step corresponding to a number of equally spaced revised dwell positions fitting the trajectory between said starting position and said finishing position and wherein the radiation dose distribution for said catheter is generated based on said equal number of revised dwell positions, said revised dwell step substantially matching the default dwell step.

According to another aspect of the present invention there is provided a radiation therapy delivery system for use in effecting radiation therapy of a pre-selected anatomical portion of an animal body, said radiation treatment system comprising:

insertion means for inserting one or more catheters into said anatomical portion, each catheter defining at least one trajectory for at least one energy emitting source;

radiation delivery means for displacing said at least one energy emitting source along said trajectory through each of said one or more catheters using a default dwell step,

along each trajectory said radiation delivery means has a defined starting position and finishing position and displaces said at least one energy emitting source through a number of equally spaced revised dwell positions fitting the trajectory between said starting position and said finishing position, wherein said revised dwell positions are determined based on the revised dwell step calculated for the default dwell step and the trajectory, said revised dwell step substantially matching the default dwell step.

According to another aspect of the present invention there is provided a method for generating a radiation treatment plan for use in effecting radiation therapy of an anatomical portion of a human or animal body, whereby one or more catheters are inserted in a certain orientation into said anatomical portion, each catheter defining a trajectory for at least one energy emitting source to be positioned at one or more dwell positions along said trajectory through said catheter using radiation delivery means, said treatment plan including information concerning;

the number and corresponding orientations of one or more of said catheters within the anatomical portion to be treated;

one or more default dwell positions in each of said one or more catheters for said at least one energy emitting source spaced apart by a default dwell step;

one or more dwell times for each of said dwell positions; and

a radiation dose distribution for each said at least one energy emitting source during its displacement along said trajectory through said one or more catheters, the method including the steps of

i) defining a starting position and a finishing position along said trajectory for each of said catheters;
ii) defining revised dwell positions corresponding to a number of equally spaced revised dwell steps fitting the trajectory between said starting position and said finishing position, said revised dwell steps substantially matching the default dwell steps; and
iii) generating the radiation dose distribution for each of said catheters based on said number of revised dwell positions.

With the method steps according to the invention the radiation dose planning system is adapted to define or redefine a starting and finishing position along said trajectory of each catheter and also to define revised dwell positions for each catheter at which the energy emitting source is to be positioned to deliver the radiation dose. For example, a following formula may be used for calculating the equally spaced revised dwell positions for a given trajectory length and the default dwell positions;

L=(d+a)×n,

wherein
L—is the length of the trajectory;
d—is the default dwell step,
n—is the number of default dwell steps necessary to cover the length of the trajectory;
a—is a real number, which must be kept minimal.

For example, for a default dwell step of 5 mm and a trajectory length of 54 mm, the revised dwell step is 5.4 mm, which ensures accurate coverage of the trajectory with a total number of 10 equally spaced steps.

It is found to be advantageous to work with the revised dwell steps and revised dwell positions as hot spots can be avoided due to lack of match between the trajectory and the first or last dwell position of the source.

Consequently it is possible to adapt the virtual working space more effectively to the (boundaries of the) anatomical portion or target (e.g. a male prostate gland or a woman's breast) and to obtain a radiation dose planning solution which provides a dose distribution profile, which more accurately matches the ideal radiation dose distribution profile position desired.

For a more accurate and ideal dose coverage of the anatomical site said starting position for each of said catheters is directly related to the position where said catheter enters said anatomical portion, and said finishing position for each of said catheters is directly related to the position where said catheter exits said anatomical portion. As a matter of normal clinical practise, primarily to avoid damage to areas outside the target volume, and to avoid damage to the skin, the starting point and the finishing point are normally about 3 mm from the boundary of the target location.

It has been proven beneficial to the treatment outcome when the starting position and the finishing position can be freely located between 1 and 7 mm of the edge or boundary of the anatomical portion (target volume).

According to another aspect of the method includes steps of

defining a dwell sub-step corresponding to a number of dwell sub-positions fitting one dwell position; and

generating the radiation dose distribution for each of said catheters based on said number of dwell sub-positions.

A further embodiment the method includes step of:

converting the dwell times for each dwell sub-position into a velocity profile for continuously displacing the energy emitting source along said dwell sub-positions of said trajectory.

It will be appreciated that the velocity profile may be obtained from a dwell time profile, as explained with reference to FIG. 5a.

With this feature an energy emitting source is not necessarily displaced in a discontinuous motion between the several dwell positions, but may be displaced in a substantially continuous motion along the trajectory of the catheter, passing through (but not necessarily stopping at) a plurality of dwell positions.

A continuous motion of the energy emitting source through the catheter can be derived from the near-continuous motion with a dwell step of 1 mm (or shorter). More particularly, a radiation treatment plan, where the energy emitting source is continuously displaced can be generated with the present invention, in an improved embodiment the method is further characterized by the steps of

modifying the length of the dwell step;

redefining correspondingly the number of dwell positions, and

recalculating the dwell times at these dwell positions in order to keep the resulting dose distribution unchanged from the original one, and

generating the radiation dose distribution for each of said catheters based on said redefined number of dwell positions.

This allows a modification of the dwell step length, based on which the radiation dose distribution for each catheter is normally determined, to a smaller or even minimized dwell step length. This reduction of the dwell step lengths along the trajectory to a minimum interval distance (approximately or even less than 1 mm) will lead to a continuous displacement of the energy emitting source without it being stopped between the dwell positions.

In an alternative embodiment of the system according to the invention, it is arranged to calculate a first portion of the trajectory for a continuous displacement of the source and a second portion of the trajectory having at least one dwell position. Such embodiment may be advantageous when an abrupt change in the velocity profile is expected.

Likewise the radiation therapy treatment planning system is according to the invention characterized in that said treatment planning means generate a treatment plan, wherein for each of said catheters a starting position and a finishing position is defined for each individual catheter. The radiation therapy treatment planning system will determine a suitable dwell step fitting the individually defined trajectory between said starting position and said finishing position and wherein the radiation dose distribution for said catheter is generated based on said dwell positions. The further reduction to about 1 mm of the length of the dwell step with the corresponding recalculation of the dwell times leads to a continuous displacement of the energy emitting source, while keeping the resulting dose distribution unchanged.

Further beneficial embodiments are described in the dependent claims.

These and other aspects of the invention will be discussed with reference to drawings, wherein like numerals refer to the like elements. It will be appreciated that the drawings are discussed for reference purpose only and may not be used for limiting the scope of the appended claims.

### BRIEF DESCRIPTION OF THE DRAWINGS

The invention will now be described in more detail with reference to the accompanying drawings, which show in:

FIG. 1a radiation therapy treatment delivery system according to the state of the art;

FIG. 2a-2b a discrete radiation dose distribution based on dwell positions and dwell times of an energy emitting source;

FIG. 3 schematically possible dwell positions of an energy emitting source along a trajectory according to the treatment planning principle of the prior art and the invention;

FIG. 4a a radiation dose distribution profile generated with a group of dwell positions according to the treatment planning principle of the prior art;

FIG. 4b a radiation dose distribution profile generated with a group of sub-dwell positions defined according to the treatment planning principle of the present invention;

FIG. 5a a dwell time profile associated to a group of sub-dwell positions defined according to the treatment planning principle of the present invention;

FIG. 5b a source velocity profile associated to the group of sub-dwell positions of FIG. 5a as defined according to the treatment planning principle of the present invention.

### DETAILED DESCRIPTION OF THE DRAWINGS

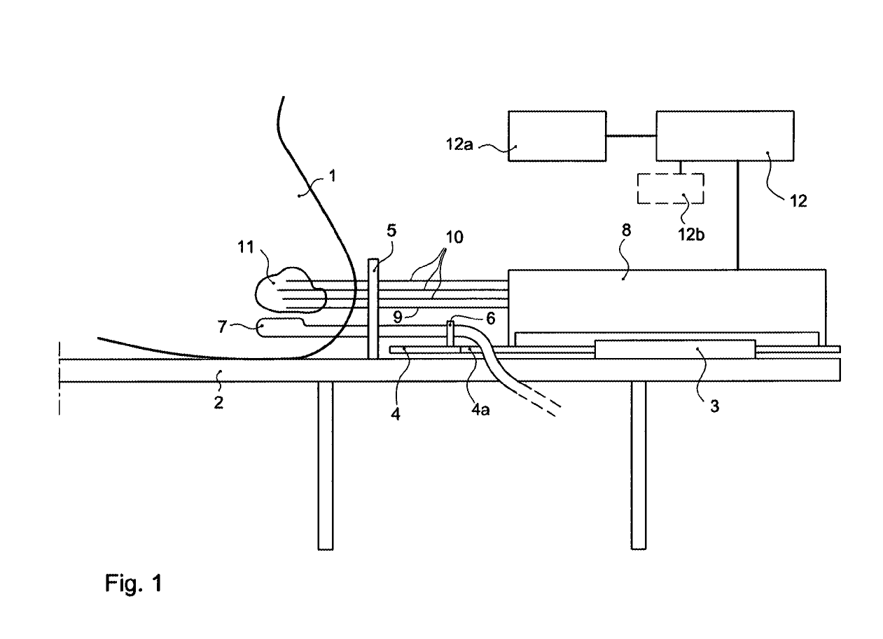

FIG. 1 shows in very schematic form various elements of a known radiation treatment delivery system for implanting an energy emitting source into a prostate gland. A patient 1 is shown lying in lithotomy position on a table 2. Fixedly connected to the table 2 is a housing 3. Housing 3 comprises a drive means 4 to move rod 4a stepwise. A template 5 is connected or mounted to the table 2, which template is provided (not shown) with a plurality of guiding holes through which holes hollow needles 9, 10 can be positioned relative to the patient. By means of a holder 6 a trans-rectal imaging probe 7 is fixedly connected to said rod 4a, which is moveable in a direction towards and from the patient by means of the drive means 4. The imaging probe 7 can be an ultrasound probe.

A needle 9 is used for fixing the prostate gland 11 in position relative to the template 5. A number of needles 10 are fixed into position through the template 5 in the prostate gland 11. The template 5 determines the relative positions of the needles 10 in two dimensions. The needles 10 are normally closed at the distal end, but may be open at their distal ends and sealed of by a plug of bio-compatible, preferably bio-absorbable wax. In said housing 3 a radiation delivery unit 8 is present.

A well-known therapy planning module 12a is provided for determining the desired number and orientation of said hollow needles as well as the relative positions of the energy emitting source(s) in each needle for displacement through said needle towards the prostate gland 11. Such therapy planning module 12a usually comprises a computer programmed with a therapy planning program. The therapy planning module 12a is connected to the radiation delivery unit 8 through a control device 12 for controlling the displacement of the one or more energy emitting sources through each needle. Control device 12 may be a separate device or may be an integrated part either of the radiation delivery unit 8 or of the therapy planning module 12a or may be embodied in the software of the therapy planning module 12a or of the radiation delivery unit 8.

The known device shown in FIG. 1 operates as follows. A patient 1 is under spinal or general anaesthesia and lies on the operating table 2 in lithotomy position. The (ultrasound) imaging probe 7 is introduced into the rectum and the probe is connected via signal line 7a with a well known image screen, where an image may be seen of the inside of the patient in particular of the prostate gland 11 as seen from the point of view of the imaging probe 7. The template 5 may be attached to the perineum of the patient to prevent or minimize any relative movement of the template and the prostate gland and the needles.

The drive means 4 is used to move the ultrasound probe longitudinally and also to rotate it to provide different angular images. The prostate gland 11 is fixed relative to the template 5 by means of one or more needles 9, 10. Subsequently further needles 10 are introduced into the body and the prostate gland one by one under ultrasound guidance.

Moving the imaging probe with the drive means 4 longitudinally and rotationally within the rectum will provide the necessary images. After all needles 10 have been placed, their positions relative to the prostate gland 11 are determined in at least one of several known ways. In a known way the therapy planning module 12a uses information from the imaging probe 7 to confirm the actual position of the treatment needles 10 and then how the one or more energy emitting sources are to be displaced through each of the needles 10. The information from the planning module 12a about the displacement of the energy emitting sources through the needles 10 in terms of dwell positions and dwell times is used to control the radiation delivery unit 8.

In the known devices energy emitting sources are moved through catheter needles in a discrete manner that is stepping motor means advance the energy emitting source in a stepwise manner between subsequent dwell positions, and the energy emitting source is maintained in each dwell position for a certain dwell time. The dwell time for each dwell position in general determines the amount of radiation delivered at each dwell position. The radiation dose at individual or particular dwell positions is to be considered as having a point source-like distribution, the peak of each radiation dose being dependent on the dwell time at said dwell position. The longer the dwell time, the higher the peak of the radiation dose at said dwell position.

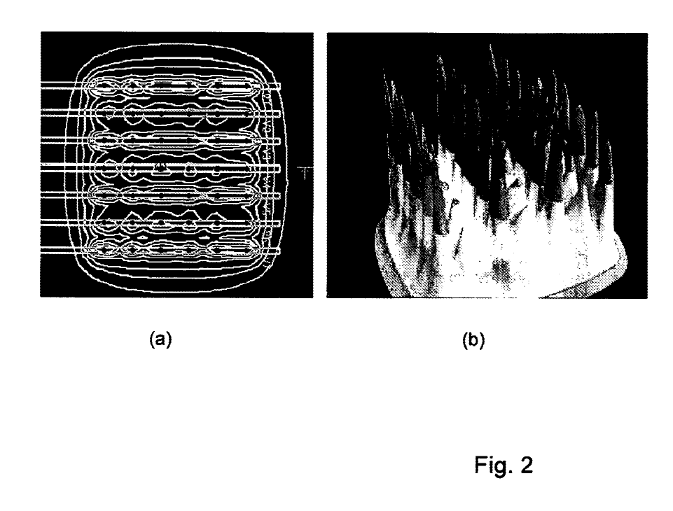

An example of a discrete radiation distribution profile resulting from the displacement of an energy emitting source through a catheter in a typical pattern of discrete dwell positions and dwell times is disclosed in FIGS. 2a and 2b. FIG. 2a shows a graphical depiction of an organ to be treated with several catheters implanted. Each catheter defines a trajectory for an energy emitting source which is to be displaced in a discrete manner and to be stopped a specific dwell positions for pre-defined dwell times.

FIG. 2b shows the radiation dose distribution of an energy emitting source at discrete dwell positions along a trajectory and around each dwell position. Processing techniques are used to smooth the peaks in the radiation dose distribution.

With the method according to the invention more advanced treatment plans can be generated, in which the treatment planning system can provide a more accurate match between the planned radiation dose distribution and the ideal radiation dose distribution profile position. This can be achieved by the choice of much smaller step sizes between the dwell points, so enabling a smoother dose distribution to be planned.

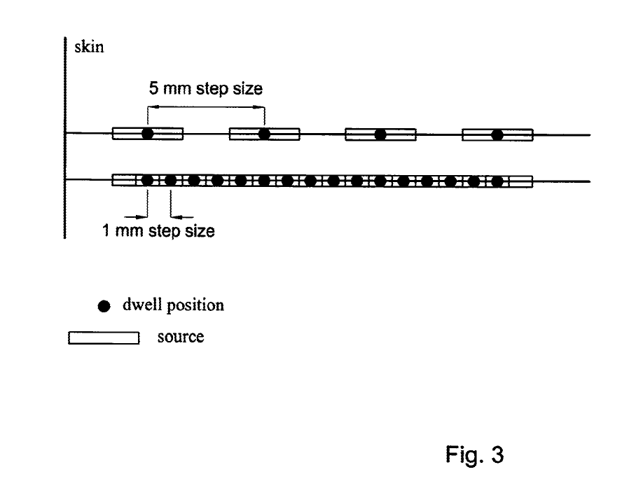

FIG. 3 depicts schematically possible dwell positions of an energy emitting source along a trajectory according to the treatment planning principle of the prior art and the invention. As is seen from FIG. 3, the source may be elongated, preferably having a length of about 1.5 mm and the dwell size may be selected to enable either a non-overlapping (5 mm) or overlapping (1 mm) mode of source displacement. It will be further appreciated that the source may have a different dimension, for example 3.7 mm or longer.

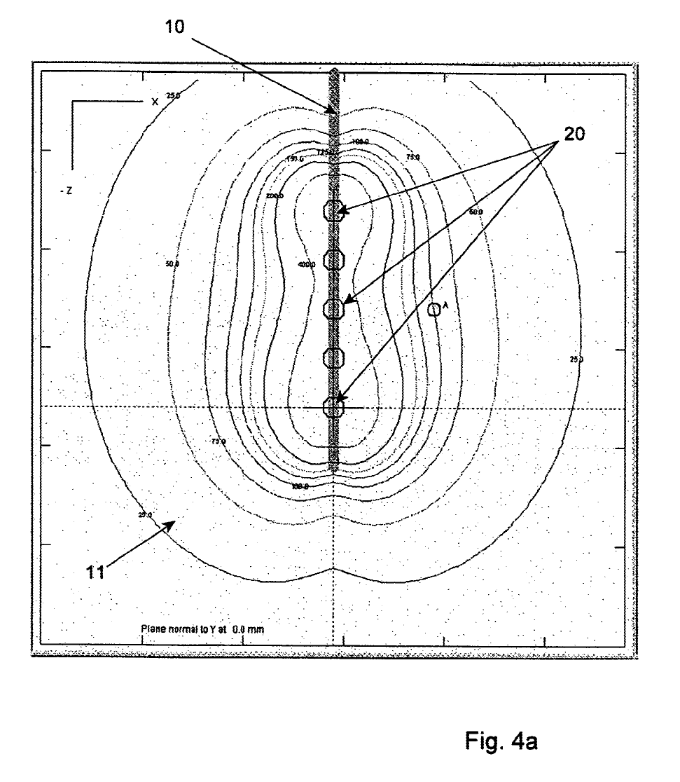

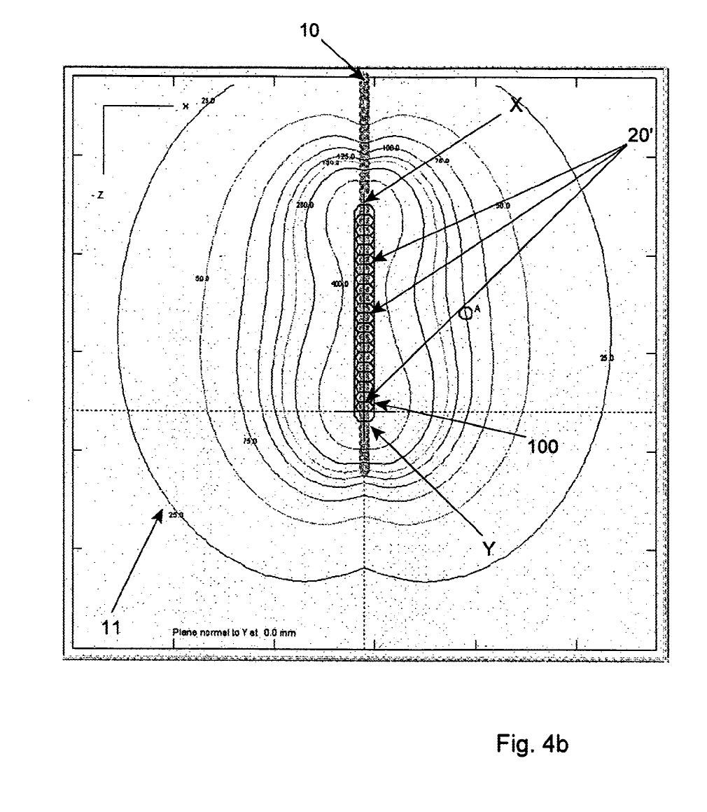

According to the invention and as disclosed in FIG. 4b the planning means define for each of said catheters 10 a starting position X and a finishing position Y along said trajectory 100. A dwell step is defined corresponding to a number of dwell positions 20′ fitting the trajectory between said starting position X and said finishing position Y and based on said number of dwell positions the radiation dose distribution for each of said catheters is generated. It is appreciated that FIG. 4a presents a dose distribution for a source displacement with a 5 mm dwell step 20.

Hence with this advanced planning technique it is now possible to generate (or calculate) radiation dose distributions which more accurately conform with the desired dose to be administrated to the target volume.

In fact by defining a starting X and finishing Y position of each catheter 10 a trajectory 100 through the anatomical portion 11 is defined, which trajectory is actually contributing to the delivery of radiation to said anatomical portion. Along said ‘actively contributing’ trajectory path new (i.e. revised) dwell positions are distributed, such that a number of dwell positions fits said ‘actively contributing’ trajectory path. This results in an optimal use of the trajectory/catheter in relation to the emission of radiation to the surrounding tissue.

The currently existing forward and inverse optimization algorithms used in the treatment planning systems remain unchanged, as they will continue to develop planning solutions based on discrete dwell positions 20 as usual. The difference with the prior art is that these dwell positions 20 are not determined by the fixed step size for all catheters, but are now determined as a function of the active length. So in other words, the length of the part of the catheter between the defined starting point and finishing point, of which a number of dwell positions 20′ fit the trajectory 100 of the catheter 10 between the pre-defined starting X and finishing Y position. The revised dwell positions are calculated based on the revised dwell step which in turn is calculated based on a default dwell step, the actual length of the trajectory and a number of steps, as explained earlier.

It is to be noted that by defining the starting X and finishing Y position for each catheter 10 the length of the trajectory 100 being used for radiation delivery may differ from the length of the trajectory 100′ of another catheter 10′. In fact, the starting X and finishing position Y according to the invention can be defined as the position where the catheter 10 should become active at the entrance to and become inactive at the exit from the target location 11. Each catheter 10-10′-10″-etc. has its own trajectory length for radiation dose delivery and also a catheter specific dwell step, a number of which fit the trajectory length of said catheter.

In other words, the distance between the first and the last dwell position is exactly equal to the active trajectory length being specified between the positions X and Y.

For a proper operation of the treatment planning means, the medical personnel must specify the active trajectory length of each catheter in the target volume, usually the starting (position X) and the ending (position Y). This is often a few mm (normally between 3-5 mm) from the boundaries of the target location or volume 11. For example with breast or skin cancers the clinical preference is not to treat an area, which is either on the surface of the organ or very close to it. This is in order to avoid—or at least minimize—the risk of damage the surrounding organs.

In the case of breast treatment care must be taken not to damage the skin. In the case of a prostate, for example, care is taken not to damage the bladder or urethra. The precise position of these other organs or structures is normally determined using image information and clinical experience.

The treatment planning means then determines the dwell positions based on said active length, the number of dwell positions which precisely fit the active trajectory length between position X and Y. Thus, each catheter 10-10′-10″ has its own revised dwell step size and the distance between the first and the last dwell position is exactly equal to the active trajectory length being specified.

In fact when treating breast cancer the starting position X and the finishing position Y are predefined by the shape and dimensions of the breast tumour and will lie several millimetres below the skin tissue in order to reduce any risk of radiation damage to the skin to a minimum level possible. Thus the active trajectory length is also preset and the dwell step length is defined as a number of steps fitting the trajectory length.

The dose distributions being calculated are based on the dwell positions being defined by the dwell step length of each trajectory which actively contributes to the radiation dose delivery. In principle, the user is not defining the dwell positions nor the dwell step length, but the active or trajectory length.

In a further improved planning system the dwell steps can be defined in an imaginary, virtual working space.

In particular these dwell steps can be reduced from a known norm or commonly used standard of about 5 mm each to a step of about 1 mm, the number of said dwell steps being calculated precisely fit the trajectory length between the defined starting position and finishing position. For example, the trajectory length could be 7.21 mm, which could be conveniently divided into seven step lengths of 1.03 mm. Likewise one dwell step length could be 1.05 mm fitting a trajectory length of 5.25 mm.

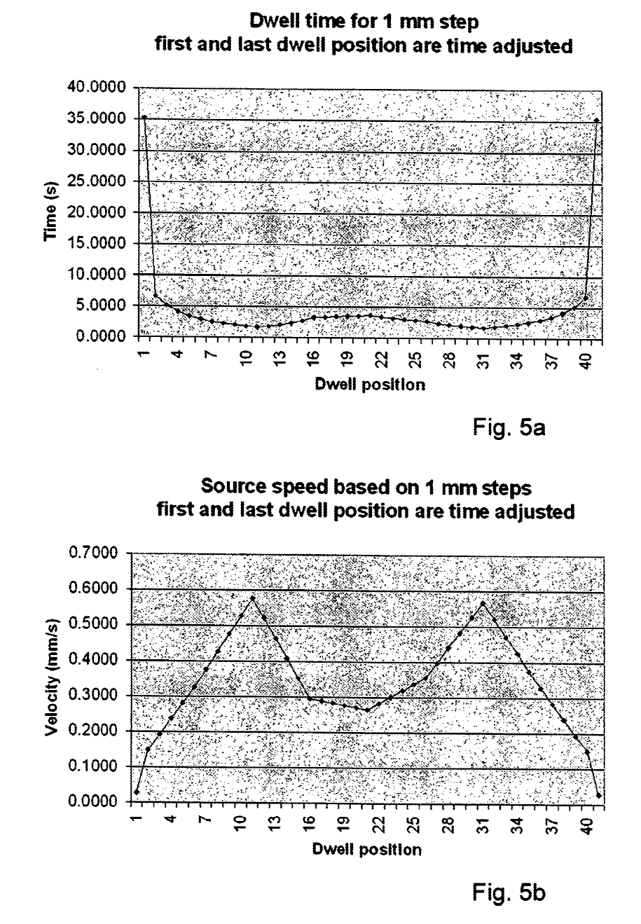

In a similar way the treatment planning software calculates for each dwell position a dwell time as depicted in FIG. 5a. Given the small dwell steps of for example 1 mm, the corresponding reduced dwell time can now be transformed into a local velocity of the energy emitting source in said dwell sub-position, defined by the source velocity as function of distance to the first dwell position. Said local velocity (in mm/sec) is defined by the inverse of the corresponding dwell time. In fact, all local velocities of the energy emitting source at the subsequent dwell positions can be converted to a velocity profile (see FIG. 5b), whereby the energy emitting source is displaced in a substantially continuous or continuous manner (instead of discrete steps as in the prior art) or in other words in a continuous motion.

During treatment planning the medical personnel can verify whether the radiation dose distribution being calculated fits the desired radiation dose distribution. In the event the pre-planned distribution is not acceptable the medical personnel may adjust a number of parameters, including for example the active trajectory length (defined by starting position X and finishing position Y) using proper inputting means, such as a computer mouse or other pointing device. The active trajectory length 100 can be adjusted by either displacing the position X and/or Y within the imaginary working space (on the planning computer), which automatically results in a different dwell step length, the number of which exactly fits the adjusted trajectory length.

The newly defined revised dwell positions (based on the redefined dwell step length) in the adjusted trajectory length are re-determined and a new radiation distribution is calculated.

When the shorter step lengths are chosen, or say approximately chosen, it is possible that the radiation dose calculations will result in very short dwell times at each dwell position. In these circumstances it is preferred to convert the step length and dwell time into a velocity profile and move the source continuously through the catheter.

Radiation delivery means described in FIG. 1 can be adapted to have short step lengths. This is relatively easily achieved by careful control of the stepper motor used in most radiation delivery means or so called after-loaders. Advantageously, such radiation treatment delivery systems can be programmed to move the energy emitting source in a continuous motion through the catheters. This continuous motion has the advantage that it is less noisy and less intrusive for the patient.

While specific embodiments have been described above, it will be appreciated that the invention may be practiced otherwise than as described. The descriptions above are intended to be illustrative, not limiting. Thus, it will be apparent to one skilled in the art that modifications may be made to the invention as described in the foregoing without departing from the scope of the claims set out below.

1. A radiation therapy treatment planning system for use in effecting radiation therapy of a pre-selected anatomical portion of a human or animal body, whereby one or more catheters are inserted in a certain orientation into said anatomical portion, each catheter defining a trajectory for at least one energy emitting source to be displaced along said trajectory through said catheter said radiation therapy treatment planning system comprising:
treatment planning means for generating a radiation treatment plan for effecting said radiation therapy, said treatment plan at least comprising information concerning:
the number, position and direction of said one or more of said catheters within said anatomical portion to be treated,
default dwell step, dwell positions and dwell times for said at least one energy emitting source along respective trajectories defined for said catheters, each trajectory having a starting position and a finishing position for each of said one or more catheters, and
a radiation dose distribution for each of said one or more catheters,
said treatment planning system including means to generate a treatment plan, arranged to determine a revised dwell step corresponding to a number of equally spaced revised dwell positions fitting the trajectory between said starting position and said finishing position and wherein the radiation dose distribution for said catheter is generated based on said equal number of revised dwell positions, said revised dwell step substantially matching the default dwell step.

2. The radiation therapy treatment planning system according to claim 1, in which image data corresponding to the anatomical portion conceived to be treated is used in said treatment planning means to help define said starting position as the position close to where said catheter enters said anatomical portion.

3. The radiation therapy treatment planning system according to claim 2, wherein said image data is used in said treatment planning means to help define said finishing position as the position close to where said catheter exits said anatomical portion.

4. The radiation therapy treatment planning system according to claim 1, wherein the starting point and/or the finishing point are between 1 and 7 mm inside the edge of the anatomical portion.

5. The radiation therapy treatment planning system according to claim 1, wherein said treatment planning system defines a dwell step corresponding to a number of equally spaced dwell positions, the system determining the radiation dose distribution for each of said catheters based on said number of dwell positions.

6. The radiation therapy treatment planning system according to claim 5, wherein the system converts the dwell times for each dwell position to a velocity profile for displacing the energy emitting source along said dwell positions of said trajectory in a continuous or substantially continuous motion.

7. The radiation therapy treatment planning system according to claim 6, wherein the system is arranged to calculate a first portion of the trajectory for continuous displacement of the source and a second portion of the trajectory having at least one dwell position.

8. The radiation therapy treatment planning system according to claim 1, wherein said radiation therapy treatment planning system further comprises inputting means for adjusting the length of the dwell step.

9. The radiation therapy treatment planning system according to claim 1, wherein the default dwell step for the default dwell positions is between 2.5-7.5 mm, preferably about 5 mm.

10. The radiation therapy planning system according to claim 1, wherein the revised dwell positions are calculated for allowing a minimal difference between the default dwell step and the revised dwell step.

11. The radiation therapy planning system according to claim 2, further comprising means for receiving said image data.

12. A radiation therapy delivery system for use in effecting radiation therapy of a pre-selected anatomical portion of an animal body, said radiation therapy delivery system comprising:
insertion means for inserting one or more catheters into said anatomical portion, each catheter defining at least one trajectory for at least one energy emitting source;
radiation delivery means for displacing said at least one energy emitting source along said trajectory through each of said one or more catheters using a default dwell step,
along each trajectory said radiation delivery means has a defined starting position and finishing position and displaces said at least one energy emitting source through a number of equally spaced revised dwell positions fitting the trajectory between said starting position and said finishing position, wherein said revised dwell positions are determined based on the revised dwell step calculated for the default dwell step and the trajectory, said revised dwell step substantially matching the default dwell step.

13. The radiation therapy delivery system according to claim 12, in which said radiation delivery means displaces said at least one energy emitting source along the trajectory between said starting position and said finishing position in a continuous motion.

14. A radiation therapy delivery system according to claim 12 in which said catheter is a hollow needle.

15. A method for generating a radiation treatment plan for use in effecting radiation therapy of an anatomical portion of a human or animal body, whereby one or more catheters are inserted in a certain orientation into said anatomical portion, each catheter defining a trajectory for at least one energy emitting source to be positioned at one or more dwell positions along said trajectory through said catheter using radiation delivery means, said treatment plan including information comprising:
the number and corresponding orientations of one or more of said catheters within the anatomical portion to be treated;
one or more default dwell positions in each of said one or more catheters for said at least one energy emitting source spaced apart by a default dwell step;
one or more dwell times for each of said dwell positions; and
a radiation dose distribution for each said at least one energy emitting source during its displacement along said trajectory through said one or more catheters, the method including the steps of
defining a starting position and a finishing position along said trajectory for each of said catheters;
defining revised dwell positions corresponding to a number of equally spaced revised dwell steps fitting the trajectory between said starting position and said finishing position, said revised dwell steps substantially matching the default dwell steps; and
generating the radiation dose distribution for each of said catheters based on said number of revised dwell positions.

16. The method according to claim 15, in which said starting position for each of said catheters is defined as the position close to where said catheter enters said anatomical portion.

17. The method according to claim 15 in which said finishing position for each of said catheters is defined as the position close to where said catheter exits said anatomical portion.

18. The method according to claim 15, wherein the starting position or the finishing position are between 1 and 7 mm from the boundary of the anatomical portion.

19. The method according to claim 15, wherein the starting position and the finishing position are defined by the user.

20. The method according to claim 15, comprising the additional steps of:
converting the dwell times for each dwell position into a velocity profile for continuously or substantially continuously displacing the energy emitting source along said dwell positions of said trajectory.

21. The method according to claim 19, wherein each of said dwell steps is between approximately 2.5 and 7.5 mm long, preferably about 5 mm long.

22. The method according to claim 19, wherein each of said dwell steps is between approximately 0.5 and 2.5 mm long.

23. The method according to claim 15, further comprising the following steps:
modifying the length of the dwell step to be equal to approximately 1 mm;
redefining correspondingly the number of dwell positions, and
recalculating the dwell times at these dwell positions in order to keep the resulting dose distribution unchanged from the original one, and
generating the radiation dose distribution for each of said catheters based on said redefined number of dwell positions.

24. The method according to claim 23, further comprising the step of defining a first portion of the trajectory for a continuous motion of the source and a second portion of the trajectory having at least one dwell position.
